(19) United States
(12) Patent Application Publication
(10) Pub. No.: US 2017/0007168 A1
(43) Pub. Date: Jan. 12, 2017

(54) METHODS AND SYSTEMS FOR PROVIDING DIAGNOSIS OR PROGNOSIS OF PARKINSON'S DISEASE USING BODY-FIXED SENSORS

(71) Applicant: THE MEDICAL RESEACH, INFRASTRUCTURE AND HEALTH SERVICES FUND OF THE TEL AVIV MEDICAL CENTER, Tel-Aviv (IL)

(72) Inventors: Anat MIRELMAN, Ramat-Gan (IL); Nir GILADE, Tel-Aviv (IL); Jeffrey M. HAUSDORFF, Hashmonaim (IL)

(73) Assignee: THE MEDICAL RESEACH, INFRASTRUCTURE AND HEALTH SERVICES FUND OF THE TEL AVIV MEDICAL CENTER, Tel-Aviv (IL)

(21) Appl. No.: 15/116,601

(22) PCT Filed: Feb. 4, 2015
(86) PCT No.: PCT/IL2015/050129

§ 371 Date: Aug. 4, 2016

Related U.S. Application Data
(60) Provisional application No. 61/935,374, filed on Feb. 4, 2014.

(51) Int. Cl. A61B 5/00; A61B 5/16; A61B 5/11

(52) U.S. Cl. CPC A61B 5/4082; A61B 5/6801; A61B 5/112; A61B 5/7278; A61B 5/4806; A61B 5/16; A61B 5/1117; A61B 5/4848; A61B 5/7267; A61B 5/7275; A61B 5/6804; A61B 5/0022; A61B 2562/0219

(57) ABSTRACT

The present disclosure relates, inter alia, to methods and systems for providing diagnosis and/or prognosis of a disease or disorder affecting movement of a subject, such as Parkinson's disease (PD), as well as determining treatment efficacy for said disorder. More particularly, the present disclosure relates, according to some embodiments, to diagnosis and/or prognosis of Parkinson's disease and/or monitoring of the disease state and/or determining or assessing treatment efficacy, using values extrapolated and/or calculated from continuous signals received by at least one Body Fixed Sensor (BFS).

### TECHNICAL FIELD

The present disclosure relates, inter alia, to methods and systems for providing diagnosis and/or prognosis of a disease or disorder affecting movement of a subject, such as Parkinson's disease (PD), as well as to systems and methods for assessing treatment efficacy of the disease or disorder. More particularly, the present disclosure relates, according to some embodiments, to diagnosis and/or prognosis of Parkinson's disease and/or monitoring of the disease state and/or monitoring treatment efficacy using values extrapolated and/or calculated from continuous signals received by at least one Body Fixed Sensor (BFS).

### BACKGROUND

Parkinson's disease (PD) is one of the most common chronic progressive neurodegenerative disorders in older adults. The incidence of PD is reported as 1%-2% of individuals ages 65 years and older worldwide. The disease also affects a large number of younger people. Patients with Parkinson's disease suffer from impairment of motor functions such as bradykinesia, rest tremor, rigidity, postural disturbances, and gait alterations, including freezing of gait (FOG) and frequent falls. Gait impairment and mobility disability are motor function impairments common in Parkinson's disease patients. These alterations in motor function, amongst which is the ability to successfully perform transitions (e.g., from sit-to-stand and stand-to-sit), are often assessed by how long it takes the participant to complete a standardized performance (e.g., Timed Up and Go, TUG). In addition to the motor functions, patients often suffer from impairment of non-motor functions such as cognitive impairment, sleep disturbances and depression.

The Unified Parkinson's Disease Rating Scale (UPDRS) is one of the most widely used instruments for measuring the severity of parkinsonian symptoms in clinical research and in practice. This standardized performance based measure includes 5 sections. The first 2 sections include a subjective assessment of non-motor aspects of the disease such as mood, swallowing and activities of daily living (ADL). Section 3 is a motor assessment that is performed by the physician and includes assessment of tremor, rigidity, movement, agility and gait. Section 4 relates to motor fluctuations and response to medications and section 5 defines the severity of symptoms. The UPDRS examination may take between 20-30 minutes or more, depending on the severity of symptoms, and requires a trained clinician to assess the patient.

In PD related clinical trials, change in the UPDRS is often the primary outcome as it enables monitoring the patient's symptoms and assessing the success of a new intervention. For example, a change of five points on the UPDRS motor part was suggested as the minimal clinically significant change. Traditionally, in patient care, the UPDRS assessment or parts of it are performed during the patient's visit to the physician's office (1-2 yearly). This time frame often presents challenges as disease progression is not always linear and subtle changes may not be readily quantified. In addition, the patient's performance during the visit to the physician's office may not accurately reflect his/her condition, in part due to ‘white coat syndrome’ or ‘reverse white coat syndrome’ (an extra effort on the part of the patient to perform well). Another, more simplified, scoring method used for prognosis of PD is the Hoehn and Yahr staging, which is used to evaluate PD state according to one of five stages.

A further obstacle in accurately assessing the disease state of Parkinson's disease patients is due to the fact that the patients suffer motor response fluctuations as the effects of anti-parkinsonian medications often wax and wane throughout the day. In the “ON” medication state, relatively soon after the patient has taken his/her medications, the patient's abilities are optimal. In contrast, in the “OFF” medication state the beneficial effects have worn out. In an attempt at capturing these “motor response fluctuations” and motor abilities in the OFF and ON medication state, the UPDRS, or at least key parts of it, is often administered in both the ON and OFF medication state. However, the UPDRS and other measures that assess symptoms are used only at one or two time points, thus not necessarily capturing the fluctuations.

WO/2013/054258 to some of the inventors discloses a method and a system for provoking gait disorders, such as freezing of gait; usable, for example, for diagnosing and/or treatment thereof.

WO/2010/150260 to some of the inventors discloses a detection of gait irregularity and/or of near fall. The publication further discloses a method of gait data collection, the method comprising collecting movement data, determining from the data a movement parameter that includes a third order derivative of position, comparing the movement parameter with a threshold value, and counting at least a near fall if the movement parameter exceeds the threshold value.

WO/2013/054257 to some of the inventors discloses methods and/or systems for diagnosing, monitoring and/or treating persons at risk for falling and/or other pathological conditions.

WO/2009/149520 discloses an automated method of determining a kinetic state of a person, the method comprising: obtaining accelerometer data from an accelerometer worn on an extremity of the person; and processing the accelerometer data to determine a measure for the kinetic state, the kinetic state being at least one of bradykinesia, dyskinesia, and hyperkinesia.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

The present disclosure provides, according to some embodiments, methods and systems for providing diagnosis and/or prognosis of a neurological disease such as Parkinson's disease (PD) in a subject based on continuous signals corresponding to body movements of the subject received by at least one body-fixed sensor (BFS). Each possibility represents a separate embodiment of the present invention.

According to a non-limiting example, a single body-fixed sensor (BFS), comprising at least one accelerometer and/or at least one gyroscope may be fixed to the trunk of a subject, typically to the lower back, and configured to receive continuous signals corresponding to the subject's body movements. The continuous signals may be acceleration signals in different axes, such as, but not limited to vertical (V), medio-lateral (ML), anterior-posterior (AP) and/or velocity in directions such as, but not limited to, yaw, pitch and roll. Each possibility represents a separate embodiment of the present invention.

According to some embodiments, a plurality of values corresponding to a plurality of motor functions and/or non-motor functions known to be affected by PD are extrapolated and/or calculated based on the continuous signals received from the subject. Each possibility represents a separate embodiment of the present invention. According to some embodiments, by comparing the calculated and/or extrapolated values to reference values, the disclosed systems and methods are able to provide diagnosis whether the subject is afflicted with PD and/or provide a prognosis as for the severity of PD in the subject. Each possibility represents a separate embodiment of the present invention. According to some embodiments, the disclosed methods and systems are configured to provide at least one quantitative measurement corresponding to the subject's diagnosis or prognosis of PD, wherein the quantitative measurement is calculated based on a plurality of the values corresponding to a plurality of motor functions and/or non-motor functions. Each possibility represents a separate embodiment of the present invention. According to some embodiments, the at least one quantitative measurement is calculated by a processor.

The present invention is based in part on the surprising discovery that various cognitive functions in a PD patient are in correlation with values calculated or extrapolated from continuous signals corresponding to the patient's body movement, as exemplified herein below. According to some embodiments, the systems and methods of the invention enable, for the first time, to evaluate both motor functions and non-motor functions in a PD patient using body movement measurements, preferably the same body movement measurements. Each possibility represents a separate embodiment of the present invention.

According to some embodiments, the disclosed method and system are configured to provide diagnosis and/or prognosis of PD in a subject based on a plurality of values calculated and/or extrapolated from the received continuous signals, wherein the plurality of values comprises at least one, preferably at least two values corresponding to motor and/or non-motor functions which are examined as part of the Unified Parkinson's Disease Rating Scale (UPDRS). Each possibility represents a separate embodiment of the present invention. According to some embodiments, the disclosed method and system are configured to provide diagnosis and/or prognosis of PD in a subject based on a plurality of values calculated and/or extrapolated from the received continuous signals, wherein the plurality of values comprises values corresponding to all motor and/or non-motor functions which are examined as part of the Unified Parkinson's Disease Rating Scale (UPDRS). According to some embodiments, the disclosed method and system are configured to provide diagnosis and/or prognosis of PD in a subject based on a plurality of values calculated and/or extrapolated from the received continuous signals, wherein the plurality of values comprises at least one, preferably at least two values corresponding to motor and/or non-motor functions which are examined as part of the Hoehn and Yahr staging. Each possibility represents a separate embodiment of the present invention.

According to some embodiments, the motor and/or non-motor functions affected by Parkinson's disease comprise at least one, preferably at least two motor and/or non-motor functions which are examined as part of the UPDRS. Each possibility represents a separate embodiment of the present invention. According to some embodiments, the motor and/or non-motor functions affected by Parkinson's disease comprise all motor and/or non-motor functions which are examined as part of the UPDRS.

According to some embodiments, the disclosed methods and systems provide a PD patient (or a subject suspected of having PD) with an accurate, quantifiable and reliable assessment of the disease state. According to some embodiments, the examined subjects can use the disclosed systems and methods in their home and community environment, as they carry out their routine activity. According to some embodiments, the disclosed methods and systems do not examine only gross motor functions, such as, but not limited to bradykinesia, but are able to measure and quantify subtle motor and/or non-motor changes that more precisely define functional deterioration and PD state. Each possibility represents a separate embodiment of the present invention.

According to one aspect, the present disclosure provides a system for providing a prognosis of Parkinson's disease in a subject, the system comprising: A body body-fixed sensor configured to receive a plurality of signals corresponding to the subject's body movement; and a processor configured to: calculate, based on the plurality of signals, a plurality of values corresponding to motor functions affected by Parkinson's disease; compare the plurality of values to a plurality of reference values; and determine the prognosis of the subject based on the comparison. According to some embodiments, the signals are continuous signals. According to some embodiments, the system comprises at least one body-fixed sensor. According to some embodiments, the system further comprises at least one sensor. According to some embodiments, the system comprises at least another sensor, such as, but not limited to, another BFS.

According to some embodiments, the present disclosure provides a system for providing a prognosis of Parkinson's disease in a subject, the system comprising: at least one sensor, wherein the at least one sensor comprises at least one body-fixed sensor configured to receive a plurality of continuous signals corresponding to the subject's body movement; and

a processor, wherein the processor is functionally connected to the at least one sensor and wherein the processor is configured to: calculate, based on the plurality of continuous signals, a plurality of values corresponding to motor functions affected by Parkinson's disease; compare the plurality of values to a plurality of reference values; and determine the prognosis of the subject based on the comparison. According to some embodiments, the disclosed system provides a subject suspected of having PD with diagnosis of PD and/or prognosis of the disease state. Each possibility represents a separate embodiment of the present invention.

According to another aspect, the present disclosure provides a method for determining prognosis of Parkinson's disease in a subject, the method comprising: receiving a plurality of signals corresponding to the subject's body movement from a body-fixed sensor; and, via a processor:

calculating, based on the plurality of signals, a plurality of values corresponding to motor functions affected by Parkinson's disease;
comparing the plurality of values to a plurality of reference values; and

determining the prognosis of the subject based on the comparison. According to some embodiments, the method comprises receiving the signals from at least one sensor.

According to some embodiments, the present disclosure provides a method for determining prognosis of Parkinson's disease in a subject, the method comprising: receiving a plurality of continuous signals corresponding to the subject's body movement from at least one sensor, wherein the at least one sensor comprises at least one body-fixed sensor functionally connected to a processor; and, via the processor:

calculating, based on the plurality of continuous signals, a plurality of values corresponding to motor functions affected by Parkinson's disease;
comparing the plurality of values to a plurality of reference values; and
determining the prognosis of the subject based on the comparison. According to some embodiments, the disclosed method provides a subject suspected of having PD with diagnosis of PD and/or prognosis of the disease state. Each possibility represents a separate embodiment of the present invention.

According to some embodiments, the body-fixed sensor is configured to be fixed to the trunk of the subject. According to some embodiments, the at least one sensor is a body-fixed sensor configured to be fixed to the trunk of the subject. According to some embodiments, the at least one sensor is a body-fixed sensor configured to be fixed to the lower back of the subject. According to some embodiments, the body-fixed sensor is configured to be fixed to the lower back of the subject. According to some embodiments, the disclosed method further comprises fixing the at least one sensor to the trunk of the subject, typically to the lower back. Each possibility represents a separate embodiment of the present invention. According to some embodiments, the disclosed method further comprises fixing the body-fixed sensor to the trunk of the subject, typically to the lower back. Each possibility represents a separate embodiment of the present invention. According to some embodiments, the at least one sensor is configured not to require recharging while receiving the signals. According to some embodiments, the body-fixed sensor is configured not to require recharging while receiving the signals.

According to certain embodiments, the disclosed systems and methods may use at least one sensor, wherein the at least one sensor is comprised within a mobile device, such as, but not limited to, a smartphone or a portable/tablet computer. Each possibility represents a separate embodiment of the present invention. According to certain embodiments, the disclosed systems and methods may use at least one sensor, wherein the at least one sensor is comprised within a mobile device and configured to receive a plurality of signals corresponding to the subject's body movements. According to some embodiments, the disclosed systems and methods may use at least one BFS and at least one sensor comprised within a mobile device.

According to some embodiments, the at least one sensor is an accelerometer. According to some embodiments, the body fixed sensor is an accelerometer. According to some embodiments, the at least one sensor comprises at least one accelerometer. According to some embodiments, the body-fixed sensor comprises at least one accelerometer. According to some embodiments, the at least one sensor comprises at least one gyroscope. According to some embodiments, the body-fixed sensor comprises at least one gyroscope.

According to some embodiments, the signals are acceleration signals. According to some embodiments, the signals comprise acceleration signals. According to some embodiments, the signals comprise velocity signals. According to some embodiments, the acceleration and/or velocity signals are in at least two axes, preferably in at least three axes, typically in at least six axes. Each possibility represents a separate embodiment of the present invention. According to some embodiments, the signals are selected from the group consisting of: vertical acceleration, medio-lateral acceleration, anterior-posterior acceleration, yaw angular velocity, pitch angular velocity, roll angular velocity and a combination thereof. Each possibility represents a separate embodiment of the present invention.

According to some embodiments, the processor is wirelessly connected to the at least one sensor. According to some embodiments, the processor is comprised in a mobile device. According to some embodiments, the mobile device is selected from the group consisting of: a mobile telephone, a portable computer, a tablet computer, a watch, a bracelet and a wearable computer. Each possibility represents a separate embodiment of the present invention.

According to some embodiments, the motor functions affected by Parkinson's disease are selected from the group consisting of: rigidity, movement amplitude, movement speed, posture, postural control, bradykinesia, gait, balance, tremor, arm swing, trunk movement, sit-to-stand transition, stand-to-sit transition, sit-to-walk transition, walk-to-sit transition, turning, sitting, lying, sleep movements and a combination thereof. Each possibility represents a separate embodiment of the present invention. According to some embodiments, motor functions affected by Parkinson's disease comprise motor functions evaluated by UPDRS. According to some embodiments, motor functions affected by Parkinson's disease comprise at least one, preferably at least two, most preferably at least five motor functions evaluated by UPDRS. Each possibility represents a separate embodiment of the present invention. According to some embodiments, the processor is configured to calculate values corresponding to at least two motor functions affected by Parkinson's disease. According to some embodiments, values corresponding to at least two motor functions affected by Parkinson's disease are calculated according to the disclosed method.

According to some embodiments, the processor is further configured to calculate, based on the comparison, at least one quantitative prognostic value corresponding to the severity of Parkinson's disease in the subject. According to some embodiments, the method further comprises calculating, based on the comparison, at least one quantitative prognostic value corresponding to the severity of Parkinson's disease in the subject. According to some embodiments, the disclosed method is configured to provide diagnosis and/or prognosis of pre-motor PD patients, which are PD patients not yet displaying impairment of motor functions and/or patients having subtle motor impairment which is non-detectable using routine methods, such as, but not limited to, the UPDRS or Hohen and Yahr methods. Each possibility represents a separate embodiment of the present invention. Without wishing to be bound by any theory or mechanism, providing diagnosis and/or prognosis for pre-motor PD patients enables to determine a suitable course of treatment for the pre-motor PD patients.

According to some embodiments, the reference values are selected from the group consisting of: values obtained from a subject having Parkinson's disease, values obtained from a healthy subject, values obtained from the subject at an earlier time period, values corresponding to Parkinson's disease of a known severity level and a combination thereof. Each possibility represents a separate embodiment of the present invention. Without wishing to be bound by any theory or mechanism, comparing the calculated values to reference values from a healthy subject may enable to diagnose PD in the subject or provide prognosis in relation to the healthy subject, comparing the calculated values to reference values from a subject having PD may enable to provide prognosis relative to the PD severity of the reference subject and comparing the calculated values to reference values obtained from the subject at an earlier time period may enable to provide prognostic information relating the disease progression in the subject. According to some embodiments, comparing the calculated values to reference values corresponding to Parkinson's disease of at least one known severity level enables determining the subject's PD severity level.

According to some embodiments, the processor is further configured to calculate, based on the plurality of signals, physiological symptoms affected by PD. According to some embodiments, the physiological symptoms affected by PD are selected from the group consisting of pain, orthostatic hypotension and a combination thereof. Each possibility represents a separate embodiment of the present invention.

According to some embodiments, the disclosed methods comprise calculating, based on the plurality of signals, a plurality of values corresponding to motor functions and/or non-motor functions and/or physiological symptoms affected by PD. Each possibility represents a separate embodiment of the present invention.

According to some embodiments, the processor is further configured to calculate, based on the plurality of signals, at least one value corresponding to at least one non-motor function affected by Parkinson's disease. According to some embodiments, the processor is further configured to calculate, based on the plurality of continuous signals, at least one value corresponding to at least one cognitive function affected by Parkinson's disease. According to some embodiments, the cognitive function is selected from the group consisting of: fatigue, sleep-pattern, global cognitive score, executive function, attention, other cognitive functions, and a combination thereof. Each possibility represents a separate embodiment of the present invention. According to some embodiments, the non-motor function comprises at least one, preferably at least two, most preferably at least three non-motor functions evaluated by the UPDRS. Each possibility represents a separate embodiment of the present invention.

According to some embodiments, a non-motor function affected by Parkinson's disease is selected from the group consisting of: a cognitive function, a sleep-behavior related function, a physiological symptom affected by PD or combinations thereof. Each possibility represents a separate embodiment of the present invention. According to some embodiments, a non-motor function affected by Parkinson's disease is a cognitive function.

According to some embodiments, the processor is further configured to compare the at least one value corresponding to at least one cognitive function to at least one reference value. According to some embodiments, the processor is further configured to determine the Parkinson's disease prognosis of the subject based on comparison of values corresponding to cognitive functions affected by Parkinson's disease with reference values. According to some embodiments, the processor is further configured to determine the Parkinson's disease prognosis of the subject based on comparison of values corresponding to motor functions and cognitive functions affected by Parkinson's disease with reference values.

According to some embodiments, the processor is further configured to compare the at least one value corresponding to at least one non-motor function to at least one reference value. According to some embodiments, the processor is further configured to determine the Parkinson's disease prognosis of the subject based on comparison of values corresponding to non-motor functions affected by Parkinson's disease with reference values. According to some embodiments, the processor is further configured to determine the Parkinson's disease prognosis of the subject based on comparison of values corresponding to motor functions and non-motor functions affected by Parkinson's disease with reference values.

According to some embodiments, the processor is configured to calculate at least part of the values corresponding to motor and/or non-motor functions affected by Parkinson's disease based on the plurality of signals collected during a specific time-window. Each possibility represents a separate embodiment of the present invention. According to some embodiments, the specific time-window is during sleep of the subject. According to some embodiments, the method of the invention comprises receiving the continuous signals and/or calculating the values during sleep of the subject. Each possibility represents a separate embodiment of the present invention.

According to some embodiments, a continuous signal is a signal received consecutively over a time period. According to some embodiments, a continuous signal is a signal received for more than 15 minutes, preferably for more than 30 minutes, most preferably for more than an hour. Each possibility represents a separate embodiment of the present invention. According to certain embodiments, a continuous signal is a signal received for more than 1 day, typically more than 3 days or 1-2 weeks. Each possibility represents a separate embodiment of the present invention.

According to some embodiments, the signals are received consecutively during both daytime and nighttime. According to some embodiments, continuous signals are received consecutively for at least one hour, for at least 4 hours, for at least 12 hours, for at least one day, for at least 3 days, for 1-2 weeks. Each possibility represents a separate embodiment of the present invention. As used herein, the term day refers to 24 hours. According to some embodiments, continuous signals are received for at least 3, preferably at least 7 days. Each possibility represents a separate embodiment of the present invention. According to some embodiments, continuous signals are received 24 hours a day for at least 1 day, preferably at least 3 days, most preferably at least 7 days. Each possibility represents a separate embodiment of the present invention. According to certain embodiments, continuous signals are received for at least 14 days, possibly for at least one month. Each possibility represents a separate embodiment of the present invention. In a non-limiting example, continuous signals are signals of acceleration in at least one axis received from a body fixed sensor for at least one day, preferably at least 3 or 7 days. Each possibility represents a separate embodiment of the present invention. According to some embodiments, the at least one sensor is configured to receive the continuous signals for at least 1 day, preferably for at least 3 days, most preferably for at least 7 days. Each possibility represents a separate embodiment of the present invention. According to certain embodiments, the at least one sensor is configured to receive the continuous signals for at least 1 hour. Without wishing to be bound by any theory or mechanism, receiving signals for a longer period of time, such as, but not limited to, at least 3 days, enables to provide more accurate calculate values, thus a more accurate diagnosis/prognosis may be achieved.

According to some embodiments, the system further comprises an output device functionally connected to the processor. According to some embodiments, the subject's body movement is selected from the group consisting of: whole body movement, trunk movement and a combination thereof. Each possibility represents a separate embodiment of the present invention. According to some embodiments, the subject's body movement is selected from the group consisting of: whole body movement, trunk movement, upper extremities movements, lower extremities movements and a combination thereof. Each possibility represents a separate embodiment of the present invention.

According to some embodiments, the disclosed method further comprises calculating, based on the comparison, at least one quantitative prognostic value corresponding to the severity of Parkinson's disease in the subject.

According to some embodiments, the disclosed method further comprises calculating, based on the plurality of continuous signals, at least one value corresponding to at least one non-motor function affected by Parkinson's disease. According to some embodiments, the disclosed method further comprises comparing the at least one value corresponding to at least one non-motor function to at least one reference value. According to some embodiments, the disclosed method further comprises determining the Parkinson's disease prognosis of the subject based on comparison of values corresponding to motor functions and/or non-motor functions affected by Parkinson's disease with reference values. Each possibility represents a separate embodiment of the present invention. According to some embodiments, calculating at least part of the values corresponding to motor or non-motor functions affected by Parkinson's disease is based on the plurality of continuous signals collected during a specific time-window, optionally during sleep of the subject.

According to yet another aspect, the present disclosure provides a method for assaying the efficiency or efficacy of a treatment for Parkinson's disease in a subject, the method comprises performing the disclosed method for determining prognosis of a PD in a subject following administration of the treatment, wherein the reference values are values corresponding to the subject prior to administration of the treatment; and determining whether the prognosis of the subject has improved. Without wishing to be bound by any theory or mechanism, the disclosed method for determining PD prognosis can be used before and after administration of treatment, wherein the reference values before treatment may be values of a healthy subject or any other predetermined reference value(s), and the reference values after treatment are the values of the examined subject prior to treatment, thereby determining whether the PD prognosis of the subject has improved following treatment relatively to the disease state prior to treatment.

According to some embodiments, the present disclosure provides a method for assaying the efficiency of a treatment for Parkinson's disease in a subject, the method comprising:
prior to administration of a treatment for PD to the subject, receiving a plurality of reference continuous signals corresponding to the subject's body movement from at least one sensor, wherein the at least one sensor comprises at least one body-fixed sensor functionally connected to a processor;
via the processor, calculating, based on the plurality of reference continuous signals, a plurality of reference values corresponding to motor functions affected by Parkinson's disease;
following administration of the treatment to the subject, receiving a plurality of continuous signals corresponding to the subject's body movement from the at least one sensor;
via the processor, calculating, based on the plurality of continuous signals, a plurality of values corresponding to motor functions affected by Parkinson's disease;
comparing the plurality of values to the plurality of reference values; and
determining the efficiency of the treatment based on the comparison.

According to some embodiments, the method for assaying the efficiency of a treatment for PD further comprises administering the treatment for PD to the subject. According to non-limiting examples, the treatment for PD may be a drug, a physical exercise, cognitive training or combinations thereof.

According to some embodiments, the processor of the disclosed system is further configured to determine a suitable course of treatment for the subject based on the comparison between calculated values and reference values. According to some embodiments, the method of the invention further comprises determining, using the processor, a suitable treatment and/or treatment regime for the subject based on the comparison between calculated values and reference values.

According to another aspect, the present disclosure provides a method for determining a cognitive state of a subject afflicted with Parkinson's disease, the method comprising:

receiving a plurality of signals corresponding to the subject's body movement from a body-fixed sensor; and, via a processor:
calculating, based on the plurality of signals, at least one value corresponding to at least one cognitive function affected by Parkinson's disease;
comparing the at least one value to at least one reference value; and

determining the cognitive state of the subject based on the comparison. According to some embodiments, the present disclosure provides a method for determining a cognitive state of a subject afflicted with Parkinson's disease, the method comprising: receiving a plurality of continuous signals corresponding to the subject's body movement from at least one sensor, wherein the at least one sensor comprises at least one body-fixed sensor functionally connected to a processor; and, via the processor:

calculating, based on the plurality of continuous signals, at least one value corresponding to at least one cognitive function affected by Parkinson's disease;
comparing the at least one value to at least one reference value; and
determining the cognitive state of the subject based on the comparison.

According to another aspect, the present disclosure provides a system for providing a prognosis of a cognitive state of a subject afflicted with Parkinson's disease, the system comprising:

a body-fixed sensor configured to receive a plurality of signals corresponding to the subject's body movement:

and

a processor configured to:
calculate, based on the plurality of signals, at least one value corresponding to at least one cognitive function affected by Parkinson's disease; compare the at least one value to at least one reference values; and determine the cognitive state of the subject based on the comparison.

According to some embodiments, the present disclosure provides a system for providing a prognosis of a cognitive state of a subject afflicted with Parkinson's disease, the system comprising:

at least one sensor, wherein the at least one sensor comprises at least one body-fixed sensor configured to receive a plurality of continuous signals corresponding to the subject's body movement;

and

a processor, wherein the processor is functionally connected to the at least one sensor and wherein the processor is configured to:
calculate, based on the plurality of continuous signals, at least one value corresponding to at least one cognitive function affected by Parkinson's disease; compare the at least one value to at least one reference values; and determine the cognitive state of the subject based on the comparison.

According to some embodiments, there is provided a system for evaluating a non-motor function affected by Parkinson's disease (PD) in a subject suffering from PD, the system comprising:
a body-fixed sensor configured to receive a signal corresponding to the subject's body movement:
and a processor configured to: calculate, based on the signal, a plurality of values corresponding to one or more motor functions affected by PD; and
evaluate the non-motor function in the subject based on the values.

In some embodiments, the non-motor function may be selected from the group consisting of: a cognitive function, a sleep-behavior related function, depressive symptoms, a physiological symptom or combinations thereof. In some embodiments, the non-motor function is a cognitive function. In further embodiments, the cognitive function is selected from the group consisting of: fatigue, sleep-pattern, global cognitive score, executive function, attention, depressive symptoms (for example, long term depression), and a combination thereof.

In some embodiments, evaluating the non-motor function may include comparing the plurality of values to a plurality of reference values. In some embodiments, the plurality of values may include vertical amplitude, stride regularity, harmonic ratio or any combination thereof.

In some embodiments, the signal may include a continuous signal. In some embodiments, the signal may include a plurality of signals.

In some embodiments, the subject's body movement may include a vertical (v) movement, an anterior posterior (AP) movement (AP), a medio-leteral (ML) movement, or any combination thereof.

In some embodiments, the signal corresponding to the subject's body movement may be selected from: vertical acceleration, medio-lateral acceleration, anterior-posterior acceleration, yaw angular velocity, pitch angular velocity, roll angular velocity or any combination thereof.

In some embodiments, the system may include two or more sensors.

In some embodiments, the body-fixed sensor may be configured to be fixed to the lower back of the subject, to the trunk of the subject, or both. In some embodiments, the body-fixed sensor may include at least one accelerometer.

In some embodiments, the processor may be wirelessly connected to the at least one sensor. In further embodiments, the processor may be comprised in a mobile device.

In some embodiments, the one or more motor functions affected by Parkinson's disease may be selected from the group consisting of: rigidity, movement amplitude, movement speed, posture, postural control, bradykinesia, gait, balance, tremor, arm swing, trunk movement, sit-to-stand transition, stand-to-sit transition, sit-to-walk transition, walk-to-sit transition, turning, sitting, lying, sleep movements or any combination thereof.

In some embodiments, the processor may be configured to calculate values corresponding to at least two motor functions affected by Parkinson's disease.

In some embodiments, the processor may further be configured to calculate, based on the comparison, at least one quantitative prognostic value corresponding to the severity of Parkinson's disease in the subject. In some embodiments, processor may further be configured to compare the plurality of values to a plurality of reference values. In some embodiments, the reference values may be selected from the group consisting of: values obtained from a subject having Parkinson's disease, values obtained from a healthy subject, values obtained from the subject at an earlier time period, values corresponding to Parkinson's disease of a known severity level or any combination thereof.

In some embodiments, the processor may be configured to calculate at least part of the values corresponding based on a signal collected during a specific time-window. In some embodiments, the specific time-window is during sleep of the subject.

In some embodiments, the system may include an output device functionally connected to the processor.

In some embodiments, the sensor may be configured to receive the signals consecutively for any period of time, such as 1-24 hours, 1-7 days, 1-4 weeks, and the like.

According to some embodiments, there is provided a method for evaluating a non-motor function affected by Parkinson's disease (PD) in a subject suffering from PD, the method comprising: receiving a signal corresponding to the subject's body movement from a body-fixed sensor; and, via a processor: calculating, based on the signal, a plurality of values corresponding to one or more motor functions affected by Parkinson's disease; and evaluating the non-motor function of the subject based on the values.

According to some embodiments, there is provided a system for determining treatment efficacy for Parkinson's disease (PD) in a subject, the system comprising:
a body-fixed sensor configured to receive a signal corresponding to the subject's body movement, wherein the signal is received prior to, during, and/or after administration of the treatment:
and a processor configured to:
calculate, based on the plurality of signals, a plurality of values corresponding to one or more motor functions affected by Parkinson's disease; compare the plurality of values to a plurality of reference values; and determine the efficacy of treatment based on the comparison. In some embodiments, the calculation and/or comparison is performed in real-time thus providing real-time feedback.

In some embodiments, the reference values may be values corresponding to the subject prior to administration of the treatment, reference values of the subject obtained at an earlier time point, reference values of a control group, reference values of subjects not afflicted with PD, reference values corresponding to Parkinson's disease of a known severity level or combinations thereof.

In some embodiments, the treatment may include a therapeutic treatment (a drug), a physical exercise, cognitive training or any combinations thereof.

In some embodiments, the signal may be a continuous signal. In some embodiments, the signal may include a plurality of signals.

In some embodiments, the system may further include at least one sensor. In some embodiments, the body-fixed sensor may be configured to be fixed to the lower back of the subject, to the trunk of the subject, or both.

In some embodiments, the body-fixed sensor may include at least one accelerometer.

In some embodiments, the signals may include acceleration signals.

In some embodiments, the signals may be selected from the group consisting of: vertical acceleration, medio-lateral acceleration, anterior-posterior acceleration, yaw angular velocity, pitch angular velocity, roll angular velocity and a combination thereof.

In some embodiments, the processor may be wirelessly connected to the sensor.

In some embodiments, the one or more motor functions affected by Parkinson's disease may be selected from the group consisting of: rigidity, movement amplitude, period of movement, movement speed, posture, postural control, bradykinesia, gait, balance, tremor, arm swing, trunk movement, sit-to-stand transition, stand-to-sit transition, sit-to-walk transition, walk-to-sit transition, turning, sitting, lying, sleep movements and a combination thereof.

In some embodiments, the system may include an output device functionally connected to the processor.

In some embodiments, the at least one sensor may be configured to receive the signals consecutively.

In some embodiments, the processor may be further configured to determine a suitable treatment regime for the subject, based on the comparison between calculated values and reference values.

According to some embodiments, there is provided a method for determining treatment efficacy for Parkinson's disease (PD) in a subject, the method comprising:
receiving a signal corresponding to the subject's body movement from a body-fixed sensor, wherein the signal is received prior to, during, and/or after administration of the treatment; and, via a processor:
calculating, based on the plurality of signals, a plurality of values corresponding to motor functions affected by Parkinson's disease:
comparing the plurality of values to a plurality of reference values; and
determining treatment efficacy based on the comparison.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below.

FIG. 1 schematically illustrates, according to certain embodiments, segments of graphs representing continuous signals received from a body fixed sensor during a sit-to-stand transition, quantifying acceleration in the V and AP axes and angular velocity in the pitch axis (Start/End indicate the start and end of the sit-to-stand transition).

FIG. 2 schematically illustrates, according to certain embodiments, segments of graphs representing continuous signals received from a body fixed sensor during a stand-to-sit transition, quantifying acceleration in the V and AP axes and angular velocity in the pitch axis (Start/End indicate the start and end of the stand-to-sit transition).

FIG. 3 schematically illustrates, according to certain embodiments, segments of graphs representing continuous signals received from a body fixed sensor during a freezing of gait (FOG) episode. The rectangle indicates the beginning and end of the FOG episode.

FIG. 4A schematically illustrates, according to certain embodiments, bar graphs comparing values calculated from continuous signals received by a body-fixed sensor placed on the lower back of PD patients with a high or low Global Cognitive Score.

FIG. 4B schematically illustrates, according to certain embodiments, bar graphs comparing values calculated from continuous signals received by a body-fixed sensor placed on the lower back of PD patients with a high or low Executive Function, which is a specific subtype of cognitive function.

FIG. 5 schematically illustrates, according to certain embodiments, segments of graphs representing continuous signals received from a body fixed sensor during sleep of a subject, quantifying acceleration in the V, ML and AP axes (arrows indicate areas which are used to calculate whether the subject is supine or lies on the right or left side).

FIG. 6 schematically illustrates, according to certain embodiments, a bar graph comparing pitch regularity calculated from continuous signals received by a body-fixed sensor placed on the lower back of PD patients which suffered from the disease for a short or long duration.

FIG. 7 schematically illustrates, according to certain embodiments, segments of graphs representing measurement of acceleration from continuous signals received from a body fixed sensor in a PD patient and a healthy subject.

FIG. 8 schematically illustrates, according to certain embodiments, a segment of graphs representing measurements of acceleration from continuous signals received from a body fixed sensor on a subject. Depicted are regions which are used to calculate values corresponding to gait, standing and sitting.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The terms “comprises”, “comprising”, “includes”, “including”, “having” and their conjugates, as used herein, mean “including but not limited to”. The terms “comprises” and “comprising” are limited in some embodiments to “consists” and “consisting”, respectively. The term “consisting of” means “including and limited to.” The term “consisting essentially of” means that the composition, method or structure may include additional ingredients, steps and/or parts, but only if the additional ingredients, steps and/or parts do not materially alter the basic and novel characteristics of the claimed composition, method or structure. In the description and claims of the application, each of the words “comprise” “include” and “have”, and forms thereof, are not necessarily limited to members in a list with which the words may be associated.

As used herein, the singular form “a”, “an” and “the” include plural references unless the context clearly dictates otherwise. For example, the term “a compound” or “at least one compound” may include a plurality of compounds, including mixtures thereof.

As used herein the term “about” refers to plus/minus 10% of the value stated. As used herein, the term “plurality” refers to at least two. According to some embodiments, the term plurality refers to more than three.

As used herein, the terms “subject”, “patient” and “subject in need thereof” are used interchangeably and refer to a subject having Parkinson's disease or a subject suspected of having Parkinson's disease.

As used herein, the term “healthy subject” refers to a subject not having Parkinson's disease and not suspected of having Parkinson's disease.

As used herein, the term “prognosis” relates to assessment of disease state at a certain time point and/or monitoring of disease advancement over a defined time period.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

According to one aspect, the present disclosure provides a system for providing a prognosis of Parkinson's disease in a subject, the system comprising:
at least one sensor, wherein the at least one sensor comprises at least one body-fixed sensor configured to receive a plurality of continuous signals corresponding to the subject's body movement; and
a processor, wherein the processor is functionally connected to the at least one sensor and wherein the processor is configured to:
   calculate, based on the plurality of continuous signals, a plurality of values corresponding to motor functions affected by Parkinson's disease; compare the plurality of values to a plurality of reference values; and determine the prognosis of the subject based on the comparison.

According to another aspect, the present disclosure provides a method for determining prognosis of Parkinson's disease in a subject, the method comprising:
receiving a plurality of continuous signals corresponding to the subject's body movement from at least one sensor, wherein the at least one sensor comprises at least one body-fixed sensor functionally connected to a processor; and, via the processor:
calculating, based on the plurality of continuous signals, a plurality of values corresponding to motor functions affected by Parkinson's disease;
comparing the plurality of values to a plurality of reference values; and
determining the prognosis of the subject based on the comparison.

According to some embodiments, the disclosed method provides a subject suspected of having PD with diagnosis of PD and/or prognosis of the disease state. Each possibility represents a separate embodiment of the present invention. According to some embodiments, the disclosed method provides diagnosis and/or prognosis of PD in the prodromal stage. Each possibility represents a separate embodiment of the present invention. According to some embodiments, the disclosed method provides diagnosis and/or prognosis of PD in a PD patient not showing motor function impairment as detected using a routine clinical examination. Each possibility represents a separate embodiment of the present invention.

According to some embodiments, the at least one sensor is a body fixed sensor (BFS). According to some embodiments, the at least one sensor comprises at least one body fixed sensor. As used herein, the terms “body-fixed sensor”, “wearable computer” and “body-worn sensor” are used interchangeably and refer to a sensor fixed to a selected location on the body of a subject (in direct or indirect contact with the skin of the subject). According to some embodiments, the at least one sensor comprises at least one sensor fixed to the trunk of the subject's body, typically on the lower back. Each possibility represents a separate embodiment of the present invention. According to some embodiments, the at least one sensor comprises at least one BFS fixed to the trunk of the subject. According to some embodiments, at least part of the continuous signals received by the at least one sensor are received by a BFS fixed to the trunk of the subject. According to some embodiments, a sensor fixed to the trunk of the subject is a sensor fixed to the lower back of the subject.

According to some embodiments, the BFS is configured to receive at least one continuous signal corresponding to the body movement of a subject wearing the BFS.

According to some embodiments, the BFS is configured to receive a plurality of continuous signals corresponding to the body movement of a subject wearing the BFS.

According to some embodiments, the at least one sensor comprises at least one sensor fixed to a wearable garment such as a shoe, shirt, belt, coat etc.

According to some embodiments, a continuous signal corresponding to the body movement of a subject is a signal corresponding to velocity/and or acceleration of at least one body part of the subject in at least one axis. Each possibility represents a separate embodiment of the present invention. According to some embodiments, the continuous signals are acceleration signals. According to some embodiments, the continuous signals are velocity signals. According to some embodiments, the continuous signals correspond to body movements selected from the group consisting of: vertical acceleration, medio-lateral acceleration, anterior-posterior acceleration, yaw angular velocity, pitch angular velocity, roll angular velocity and a combination thereof. Each possibility represents a separate embodiment of the present invention.

According to some embodiments, the at least one sensor comprises at least one accelerometer. According to some embodiments, the at least one BFS comprises at least one accelerometer. According to some embodiments, the at least one sensor is an accelerometer. According to some embodiments, the at least one BFS is an accelerometer. According to some embodiments, the at least one sensor comprises at least one gyroscope. According to some embodiments, the at least one BFS comprises at least one gyroscope.

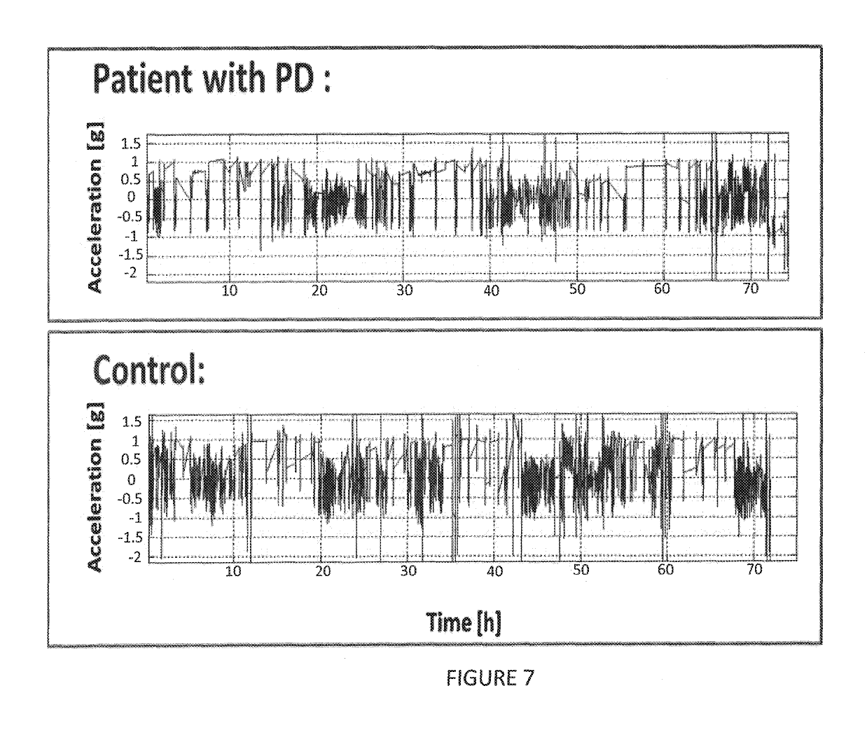

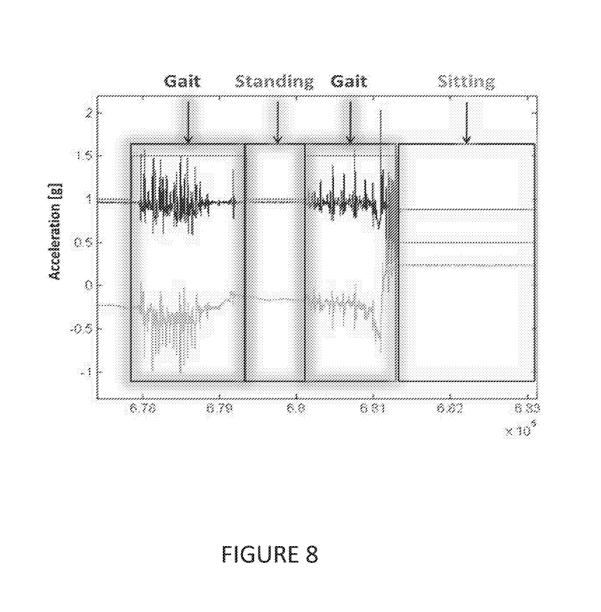

Reference is now made to FIG. 7, schematically illustrating, according to some embodiments, continuous acceleration signal obtained from a single 3-axis accelerometer worn for 72 hours on the lower back of a patient with PD and a control patient who was age matched with the PD patient. FIG. 8 schematically illustrates, according to some embodiments, processing of the signal depicted in FIG. 7 to arrive at characterization of various motor functions known to be affected by Parkinson's disease such as gait, standing, sitting and the transitions between these functions. According to some embodiments, a processor, as in the disclosed systems and methods is configured to quantify these functions and calculate a plurality of values corresponding to the functions, such as, but not limited to, values corresponding to the amount of time spent in each function, the quality of each movement and the like.

According to some embodiments, the at least one sensor is configured to be fixed to the trunk of the subject. According to some embodiments, the at least one BFS is configured to be fixed to the trunk of the subject. According to some embodiments, the disclosed method comprises fixing the at least one sensor to the trunk of the subject. According to some embodiments, the disclosed method comprises fixing the at least one BFS to the trunk of the subject. According to some embodiments, the at least one sensor is configured to be fixed to the lower back of the subject. According to some embodiments, the at least one BFS is configured to be fixed to the lower back of the subject. According to some embodiments, the disclosed method comprises fixing the at least one sensor to the lower back of the subject. According to some embodiments, the disclosed method comprises fixing the at least one BFS to the lower back of the subject.

According to some embodiments, the at least one sensor is functionally connected to the processor. According to some embodiments, the at least one sensor is configured to transfer the continuous signals to the processor.

According to some embodiments of the present disclosure, the disclosed system comprising at least one body-fixed sensor (BFS) or an array of body-fixed sensors is configured to assess parkinsonian symptoms and their changes over time. According to some embodiments, the disclosed system provides a subject suspected of having PD with diagnosis of PD and/or prognosis of the disease state. Each possibility represents a separate embodiment of the present invention.

According to some embodiments, a BFS can collect data, unobtrusively and continuously, over an extended period of time, such as, but not limited to, hours, days, weeks or months. Each possibility represents a separate embodiment of the present invention. According to some embodiments, the disclosed systems and methods enable to use continuous signals obtained from at least one sensor to quantify and/or characterize parkinsonian symptoms and/or disease progression and/or response to medication or therapeutic interventions (e.g., exercise, deep brain stimulation). Each possibility represents a separate embodiment of the present invention.

According to some embodiments, the disclosed systems and methods enable continuous monitoring of motor and/or non-motor functions affected in Parkinson's disease using at least one sensor, typically at least one BFS. Each possibility represents a separate embodiment of the present invention. According to some embodiments, continuous monitoring of motor and/or non-motor functions affected in Parkinson's disease using the at least one sensor allows for quantitative assessment of a plurality of the functions. According to some embodiments, continuous monitoring of motor and/or non-motor functions affected in Parkinson's disease using the at least one sensor allows for quantitative assessment of a plurality of the functions in parallel.

According to some embodiments, continuous monitoring of motor and/or non-motor functions affected in Parkinson's disease using the at least one sensor allows for assessment and/or quantification of a plurality of functions comprising functions which are otherwise assessed subjectively by self-reports of patients or their care givers. Such functions include, but are not limited to, activity of daily living (ADL), sleep patterns and endurance. According to some embodiments, continuous monitoring using at least one BFS according to the disclosed methods and systems allows to identify fluctuations in motor and/or non-motor functions associated with Parkinson's disease which occur during the day, thus providing an accurate and quantitative measurement of Parkinson's disease symptoms at a certain time point or time period. Each possibility represents a separate embodiment of the present invention. In a non-limiting example, as exemplified herein below, the disclosed systems and methods are able to monitor functions such as, but not limited to, sit-to-stand transition, stand-to-sit transition, sleep behavior, executive function and global cognitive score and their changes in time. According to some embodiments, the disclosed methods and systems enable determining PD prognosis and/or diagnosis of a subject by continuous monitoring a plurality of motor and/or non-motor functions which are known to be affected by PD.

According to some embodiments, the disclosed system is portable. According to some embodiments, the system of the present invention is configured for home and/or community living and/or clinical use. Each possibility represents a separate embodiment of the present invention. According to some embodiments, the at least one BFS is configured to be continuously fixed to the body of the subject. According to some embodiment, the subject may use the system of the invention in a home or community setting, thus obviating the need for a care giver in order to administer assays which characterize PD symptoms.

According to some embodiments, the disclosed systems and methods are configured to provide diagnosis and/or prognosis of PD based on an objective and comparable measurement and independent of the judgment of the subject and/or care giver. Each possibility represents a separate embodiment of the present invention. According to some embodiments, measuring motor and/or non-motor functions using continuous signals received by at least one BFS can provide objective and sensitive measures that can be obtained without the patient having to arrive at the clinic. According to some embodiments, the system further comprises an element capable of transmitting the calculated values and/or continuous signal to a distant computer, typically to the computer of a treating physician.

According to some embodiments, the at least one sensor, typically at least one BFS, is functionally connected to a processor which receives the continuous signals from the at least one sensor. According to some embodiments, the at least one sensor is physically connected to the processor. According to some embodiment, the sensor is wirelessly connected to the processor. According to some embodiments, the processor is comprised in a mobile device, such as, but not limited to a mobile telephone, a portable computer, a tablet computer and the like, and the sensor wirelessly transmits the continuous signals to the processor. According to some embodiments, the system of the invention further comprises a storage element functionally connected to the processor. According to some embodiments, the storage element is selected from the group consisting of: physical storage element, cloud-type storage element and a combination thereof. Each possibility represents a separate embodiment of the present invention. According to some embodiments, the processor is configured to constantly store at least part of the calculated values and/or continuous signals on the storage element. Each possibility represents a separate embodiment of the present invention. According to some embodiments, the storage element is functionally connected to the at least one sensor. According to some embodiments, the storage element is configured to store at least part of the continuous signals received by the at least one sensor and/or at least part of the values calculated by the processor based on the continuous signals. Each possibility represents a separate embodiment of the present invention. According to some embodiments, the disclosed method further comprises storing at least part of the continuous signals received by the at least one sensor and/or at least part of the values calculated by the processor in the storage element. According to some embodiments, the processor is further configured to retrieve stored continuous signals and/or stored values and use them to provide an assessment of PD diagnosis and/or prognosis for a specific time point. Each possibility represents a separate embodiment of the present invention. According to some embodiments, the processor is further configured to retrieve stored continuous signals and/or stored values and use them to calculate reference values that would be used according to the disclosed method and system at a future time point by the same or different user. Each possibility represents a separate embodiment of the present invention.

According to some embodiments, the processor is configured to calculate a plurality of values corresponding to motor and/or non-motor functions affected by Parkinson's disease, based on the continuous signals received by at least one sensor, typically at least one BFS. Each possibility represents a separate embodiment of the present invention. According to some embodiments, the values are calculated based on a plurality of continuous signals. In a non-limiting example, as exemplified herein below, calculating a value corresponding to a subject's sleep behavior can be achieved based on continuous signals measuring acceleration in the vertical medio-lateral and anterior-posterior axes.

According to some embodiments, a single value or a plurality of values may be calculated to characterize a certain motor or non-motor function. In a non-limiting example, to characterize sleep behavior values may be calculated to determine measures such as, but not limited to, times the subject got up, number of disruptions, restlessness/movement, frequency of disruptions, nocturnia and the like.

According to another non-limiting example, a plurality of values may be calculated to characterize gait, such as, but not limited to the number of steps per day, number of walking bouts, the number of walking bouts over of a minimal duration, average, range, variability, and longest walking bout, stride length, gait speed, gait asymmetry, and gait variability. In addition, values corresponding to changes within a given walking bout as well as values corresponding to the distribution and changes over time may be calculated.

According to some embodiments, values are calculated by the processor through extrapolation of characteristics of graphs corresponding to a plurality of continuous signals received by at least one BFS. According to some embodiments, the processor is further configured to calculate a pattern from the plurality of calculated values, compare the pattern to a pattern calculated from a plurality of reference values and determine the prognosis of the subject based on the comparison.

According to some embodiments, the processor according to the disclosed methods and systems determines a prognosis of Parkinson's disease state based on a comparison between the calculated plurality of values to a plurality of reference values. According to some embodiments, the reference values are values corresponding to at least one PD patient having a known disease state. According to some embodiments, comparing the calculated values to reference values corresponding to at least one PD patient having a known disease state enables to determine the disease state of the subject in related to the subject or subjects from which the reference values were derived. According to some embodiments, the reference values are valued from the same subject, measured at an earlier time point. According to some embodiments, comparing the calculated values to reference values measured from the same subject at an earlier time point enables determining whether there is an improvement or deterioration in the PD state of the subject or in a certain aspect of the disease, such as, but not limited to, executive function. According to other embodiments, comparing the calculated values to reference values measured from the same subject at an earlier time point enables determining whether a treatment administered to the subject in between measurement of values resulted in an improvement in the subjects PD state or part thereof. According to some embodiments, the disclosed methods enable determining the efficiency of a treatment for PD by administering the method of the invention before and after the treatment and comparing the values calculated, wherein values indicating on an improved PD diagnosis indicate the efficiency of the treatment. It is to be noted that, according to some embodiments of the invention, the disclosed system and method may be used to calculate values corresponding to motor and/or non-motor functions based on continuous signals and store the values in a storage element without comparing to reference values and determining a PD prognosis. Such stored values may be used as reference values when the same or different subject uses the disclosed system or method at a later time point.

According to some embodiments, the reference values are values measured from a healthy subject. According to some embodiments, comparing the calculated values to reference values measured from a healthy subject enable to provide diagnosis whether the subject is suspected to have PD and/or provide prognosis of the disease severity. Each possibility represents a separate embodiment of the present invention.

According to some embodiments, comparing the calculated values to reference values enables to assess the severity of at least one aspect of PD symptoms in the subject, such as, but not limited to, tremor, gait impairment and cognitive impairment. Each possibility represents a separate embodiment of the present invention. According to some embodiments, the disclosed systems and methods are configured to determine the prognosis of the subject based on comparison of values corresponding to motor function, non-motor functions or a combination thereof. Each possibility represents a separate embodiment of the present invention. According to some embodiments, the disclosed systems and methods are configured to determine the prognosis of the subject based on comparison of a plurality of values corresponding to motor functions, typically motor functions which are routinely examined as part of the UPDRS. Each possibility represents a separate embodiment of the present invention. According to some embodiments, the disclosed systems and methods are configured to determine the prognosis of the subject based on comparison of a plurality of values corresponding to motor functions, typically motor functions which are routinely examined as part of the Hoehn and Yahr staging. Each possibility represents a separate embodiment of the present invention.

According to some embodiments, the processor is configured to produce at least one quantitative value corresponding to the subjects PD prognosis as determined by the processor. According to some embodiments, the at least one quantitative value corresponds to at least one value as examined as part of the UPDRS.

According to some embodiments, the calculated values may correspond to values which are measured by traditional UPDRS thus creating estimates of the state of classic PD motor symptoms. In a non-limiting example, stride length, extrapolating gait speed and transition features from the continuous signals received by the at least one BFS may be used to calculate a value corresponding to bradykinesia. Similarly, a value corresponding to tremor may be calculated by extrapolating sitting and standing information from the continuous signals. A value corresponding to axial rigidity may be estimated by measuring signals corresponding to walking and transitions. Postural control can be estimated by extrapolating data related to standing and walking bouts from the continuous signals. Thus, calculated values relating to motor features extracted from the continuous signals received by at least one BFS may provide an accurate and automated assessment corresponding to the motor part II of the UPDRS. In addition, key non-motor functions affected in PD, such as cognitive function and sleep, may also be evaluated. Estimates of the range, distribution, minimum, maximum and related metrics of the continuous signals may be used to estimate fluctuations in each of these different aspects of function, motor response fluctuations, and best and worse performance (i.e., ON, OFF medications) reflecting information that is featured in section IV of the UPDRS.

According to some embodiments, the processor according to the disclosed methods and system calculated and compares a plurality of values usually determined by the UPDRS, such as, but not limited to: tremor, rigidity, movement amplitude, posture, bradykinesia, gait, balance, daily activity, ADL, sleep pattern, fatigue, cognitive function or combinations thereof. Each possibility represents a separate embodiment of the present invention. According to some embodiments, sleep pattern comprises sleep behavior such as, but not limited to: nocturia, sleep architecture, number and/or frequency of getting up during the night, body position during sleep or combinations thereof. Each possibility represents a separate embodiment of the present invention.

According to some embodiments, the present disclosure provides a method for prognosis of sleep and/or monitoring of sleep in a subject, the method comprising:

receiving a plurality of continuous signals corresponding to the subject's body movement during sleep from at least one sensor, wherein the at least one sensor comprises at least one body-fixed sensor functionally connected to a processor; and, via the processor:
calculating, based on the plurality of continuous signals, a plurality of values corresponding to motor and/or cognitive functions occurring during sleep:
comparing the plurality of values to a plurality of reference values; and determining the prognosis of the subject based on the comparison. Each possibility represents a separate embodiment of the present invention. According to some embodiments, cognitive functions occurring during sleep include, but are not limited to, nocturia, sleep architecture, number and/or frequency of getting up during the night, body position during sleep or combinations thereof. Each possibility represents a separate embodiment of the present invention. According to some embodiments, the disclosed systems and methods may be used to monitor and/or provide prognosis on the sleep quality of a subject by receiving continuous signals from at least one BFS during sleep of a subject and comparing the values calculated from the signals with reference values of a subject not having sleep interference or the same subject at a different time point. Each possibility represents a separate embodiment of the present invention.

According to some embodiments, the disclosed system further comprises an output device functionally connected to the processor. The output device may provide visual and/or audible signals and/or any form of sensory feedback, such as, tactile signal. According to some embodiments, the processor is further configured to display at least part of the continuous signals and/or at least part of the calculated values and/or graphic representations thereof and/or the determined prognosis or a graphic representation thereof on the output device. Each possibility represents a separate embodiment of the present invention. According to some embodiments, the processor is wirelessly connected to the output device. According to some embodiments, the disclosed methods further comprise displaying on an output device functionally connected to an output device at least part of the continuous signals and/or at least part of the calculated values and/or graphic representations thereof and/or the determined prognosis or a graphic representation thereof on the output device. Each possibility represents a separate embodiment of the present invention.

According to some embodiments, the processor is further configured to determine a treatment regime corresponding to the prognosis of the subject. According to some embodiments, the method further comprises determining, using the processor, a treatment regime corresponding to the prognosis of the subject.

According to some embodiments, the disclosed systems and methods may be used to provide diagnosis and/or prognosis and/or monitoring of other diseases or conditions which affect various motor and/or non-motor functions, such as, but not limited to atypical parksinonism, myasthenia gravis, multiple sclerosis, post-stroke symptoms and dementia. According to some embodiments, using the disclosed systems and methods enables to monitor motor and/or non-motor functions over time and this provides diagnosis and/or prognosis in patients afflicted with a disease or disorder affecting movement of a subject, such as, but not limited to, PD, atypical parksinonism, myasthenia gravis, multiple sclerosis, or post stroke symptoms. Each possibility represents a separate embodiment of the present invention.

According to some embodiments, there is provided a system for evaluating a non-motor function affected by Parkinson's disease (PD) in a subject suffering from PD, the system comprising a body-fixed sensor configured to receive a signal corresponding to the subject's body movement; and a processor configured to: calculate, based on the signal, a plurality of values corresponding to one or more motor functions affected by PD; and evaluate the non-motor function in the subject based on the values.

According to some embodiments, there is provided a method for evaluating a non-motor function affected by Parkinson's disease (PD) in a subject suffering from PD, the method comprising: receiving a signal corresponding to the subject's body movement from a body-fixed sensor; and, via a processor: calculating, based on the signal, a plurality of values corresponding to one or more motor functions affected by Parkinson's disease; and evaluating the non-motor function in the subject based on the values

According to some embodiments, there is provided a system for determining treatment efficacy for Parkinson's disease (PD) in a subject, the system comprising: a body-fixed sensor configured to receive a signal corresponding to the subject's body movement, wherein the signal is received prior to, during, and/or after administration of the treatment; and a processor configured to: calculate, based on the plurality of signals, a plurality of values corresponding to one or more motor functions affected by Parkinson's disease; compare the plurality of values to a plurality of reference values; and determine the efficacy of treatment based on the comparison.

According to some embodiments, there is provided a method for determining treatment efficacy for Parkinson's disease (PD) in a subject, the method comprising: receiving a signal corresponding to the subject's body movement from a body-fixed sensor, wherein the signal is received prior to, during, and/or after administration of the treatment; and, via a processor: calculating, based on the plurality of signals, a plurality of values corresponding to motor functions affected by Parkinson's disease; comparing the plurality of values to a plurality of reference values; and determining treatment efficacy based on the comparison. In some embodiments the calculation and/or comparison may be preformed in real-time, such that a feedback indication regarding the treatment efficacy is received in real time. In some embodiments, the method may further include determining a suitable treatment regime for the subject, based on the comparison between calculated values and reference values.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

### EXAMPLES

### Example 1

### Measuring Body Movements Using a Body-Fixed Sensor Enables Distinguishing Parkinson's Disease Patients (PD) from Other Subject Groups

In order to evaluate whether measurement of body movements using a body-fixed sensor may be used similarly to a Timed Up and Go (TUG) test, typically administered in the lab or clinic under well-defined conditions to identify subclinical gait impairments and/or determine which aspects of mobility are impaired, acceleration data was collected during three days using a small device attached to the lower back of subjects with Parkinson's disease (PD), older adults (OA) and idiopathic fallers (FL). The experiment presented herein below focused on transitions from standing to sitting, and vice versa, which are an important part of the TUG test. An algorithm was developed to identify and analyze the transitions for each subject during unconstrained every day activity.

Subjects wore a small device (DynaPort Hybrid, McRoberts, The Hague, Netherlands; 87×45×14 mm, 74 g) that contained accelerometers and gyroscopes on the lower back, approximately at the level of L4-5. Six channels were collected at 100 Hz each: vertical (V) acceleration, medio-lateral (ML) acceleration, anterior posterior (AP) acceleration, and angular velocity in three directions: yaw, pitch and roll. The subjects wore the sensor for three consecutive days.

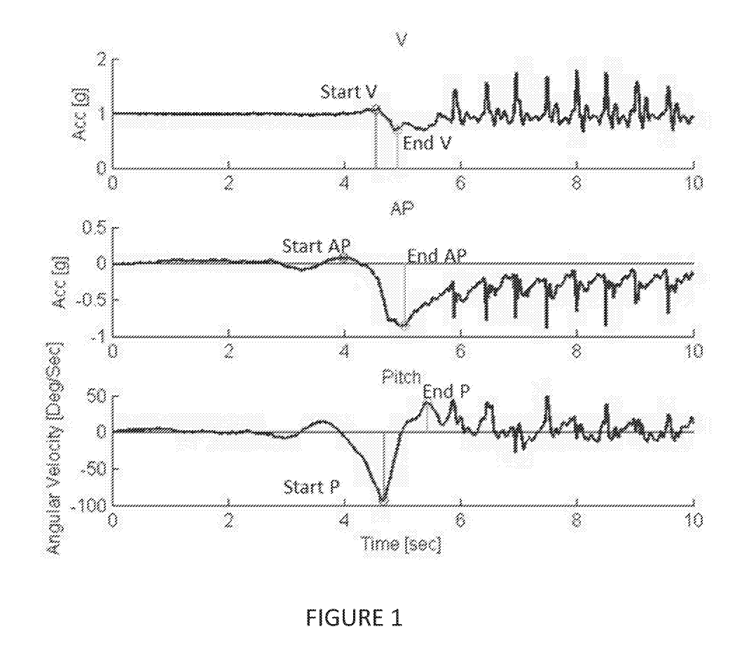

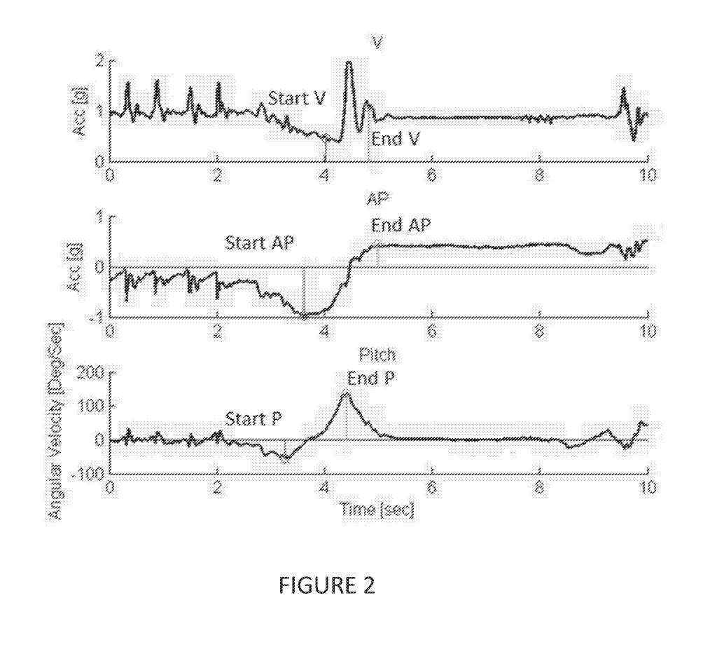

In order to find the transitions that the subject preformed during the day, the standing and sitting segments were first detected in the data using the mean of the AP signal. A low mean AP signal is mostly result from standing and high mean AP is mostly results from sitting. The gait was further identified in the standing segments. Then, windows of 10 seconds around the transitions points were looked at. In order to reduce noise we demanded that the transitions will comply with 3 conditions: pitch range was above 15 [deg/sec], the change in the AP range between the mean of the first half and the mean of the second half of the transition window was above 10.31 [g] and the range of the sitting part of the window was below 0.4 [g]. From the 10 seconds windows that complied with these 3 conditions the start and end point of the transitions were found and their duration [msec], range jerk [g] and standard deviation (STD) [g] were extracted in 3 axes—V, AP, and pitch. FIGS. 1 and 2 demonstrate a typical sit-to-stand and stand-to-sit transitions, respectively, including their start and end points. Each axis in FIGS. 1-2 expresses a different aspect of the movement hence the different start and end points.

The significance of each feature of the signals measured by the sensor was examined on its own and, additionally, machine learning algorithms were used to examine the ability of the entire feature set together to distinguish between groups of PD, OA and FL subjects. Machine learning is an approach to design and develop algorithms which use empirical input and provide predictions of the underlying mechanisms that generated the input. Our algorithms built decision trees using four common methods: ‘Ada Boost’, ‘SVM’, ‘Bag’ and ‘Naïve Bayes’. The algorithms were used to try and distinguish the PD from the OA and the FL from the OA. The test group for the PD-OA was built from 25 subjects from each group and for the FL-OA from 20 subjects from each group. The subjects for the test group were randomly chosen and the testing part was performed on the rest. Since the results are affected by the test set, 20 iterations were used, and the results represent the mean value.

Out of the 173 subjects which participated in the experiment, 102 were PD patients (27 females; age 64.8+\−9.3 yrs; disease duration 5.4+\−3.4 yrs), 33 were FL subject (22 females; age 77.8+\−4.9) and 38 were OA subjects (24 females; age 8.6+\−4.3) used as control. A subject was classified as a faller if he/she reported at least two falls in the last year.

In total, 9919 stand to sit transitions were collected, of which only 4865 contained gait in the stand part (average of 28.1 per subject), and 9757 sit to stand transitions of which only 5054 (average of 29.2 per subject) contained gait in the stand part. The rate of stand-to-sit transitions lacking gait from the total stand-to-sit transitions was significantly different between the FL and the OA groups (p=0.02). Transitions which had problematic start or end point were extracted from the analysis. In total, 4025 (average of 23.2 per subject) stand to sit transitions and 3527 (average of 20.3 per subject) sit to stand transitions were analyzed. Table 1 depicts the features calculated from the signals received by the sensor in each axis and for each transition (Range—relates to range of acceleration within examined axis, Duration—relates to acceleration time, Jerk—relates to the Range's derivative, STD—relates to the standard deviation of the Range). Table 2 depicts the measurements from each examined group.

TABLE 1
Features measured by sensor

| | Stand | Sit to | | |
|---|---|---|---|---|
| Features name | to sit | stand | axis | Feature explanation |
| TrangeV | + | − | V | Range [g] |
| TtimeV | + | − | V | Duration [msec] |
| TjerkV | + | − | V | Jerk [g/msec] |
| TstdV | + | − | V | STD [g] |
| TrangeAP | + | − | AP | Range [g] |
| TtimeAP | + | − | AP | Duration [msec] |
| TjerkAP | + | − | AP | Jerk [g/msec] (Range |
| | | | | derivative) |
| TstdAP | + | − | AP | STD [g] (of Range) |
| TrangeP | + | − | Pitch | Range [g] |
| TtimeP | + | − | Pitch | Duration [msec] |
| TjerkP | + | − | Pitch | Jerk [g/msec] |
| TstdP | + | − | Pitch | STD [g] |
| TtimeAf | + | − | V | The area below the |
| | | | | histogram's of the CDF |
| | | | | of the duration |
| TtimeMax_hNorm | + | − | V | The present of data in the |
| | | | | highest peak of the |
| | | | | histogram of the duration |
| TstdVAf | + | − | V | The area below the |
| | | | | histogram's of the CDF |
| | | | | of the STD |
| TstdVMax_hNorm | + | − | V | The present of data in the |
| | | | | highest peak of the |
| | | | | histogram of the STD |
| TransitionTypeToSit | + | − | All | The percent of gait to sit |
| | | | | transition from the over- |
| | | | | all transition |
| lengthRangeVToSit | + | − | All | The number of analyzed |
| | | | | transition |
| FrangeV | − | + | V | Range [g] |
| FtimeV | − | + | V | Duration [msec] |
| FjerkV | − | + | V | Jerk [g/msec] |
| FstdV | − | + | V | STD [g] |
| FrangeAP | − | + | AP | Range [g] |
| FtimeAP | − | + | AP | Duration [msec] |
| FjerkAP | − | + | AP | Jerk [g/msec] |
| FstdAP | − | + | AP | STD [g] |
| FrangeP | − | + | Pitch | Range [g] |
| FtimeP | − | + | Pitch | Duration [msec] |
| FjerkP | − | + | Pitch | Jerk [g/msec] |
| FstdP | − | + | Pitch | STD [g] |
| FrangePitchAf | − | + | Pitch | The area below the |
| | | | | histogram's of the CDF |
| | | | | of the range |
| FrangePitchMax_hNorm | − | + | Pitch | The present of data in the |
| | | | | highest peak of the |
| | | | | histogram of the range |
| FstdPitchAf | − | + | Pitch | The area below the |
| | | | | histogram's of the CDF |
| | | | | of the STD |
| FstdPitchMax_hNorm | − | + | Pitch | The present of data in the |
| | | | | highest peak of the |
| | | | | histogram of the STD |
| TransitionTypeFromSit | − | + | All | The percent of sit to gait |
| | | | | transition from the over- |
| | | | | all transition |
| lengthRangeVFromSit | − | + | All | The number of analyzed |
| | | | | transition |

TABLE 2
Measurements taken in each examined group

| | OA | | FL | | PD | |
|---|---|---|---|---|---|---|
| | | Std. | | Std. | | Std. |
| Feature Name | Mean | Deviation | Mean | Deviation | Mean | Deviation |
| TrangeV | 0.44 | 0.10 | 0.42 | 0.14 | 0.34 | 0.12 |
| TtimeV | 55.63 | 19.59 | 58.20 | 20.24 | 62.71 | 25.73 |
| TjerkV | 0.01 | 0.00 | 0.01 | 0.00 | 0.01 | 0.00 |
| TstdV | 0.14 | 0.04 | 0.16 | 0.04 | 0.13 | 0.05 |
| TrangeAP | −0.96 | 0.16 | −0.94 | 0.15 | −0.82 | 0.17 |
| TtimeAP | 128.55 | 23.25 | 139.95 | 31.79 | 146.64 | 31.41 |
| TjerkAP | −0.01 | 0.00 | −0.01 | 0.00 | −0.01 | 0.00 |
| TstdAP | 0.32 | 0.05 | 0.31 | 0.05 | 0.26 | 0.06 |
| TrangeP | 128.39 | 26.55 | 176.31 | 108.99 | 103.31 | 38.04 |
| TtimeP | 100.05 | 17.33 | 107.52 | 18.87 | 102.85 | 18.37 |
| TjerkP | 1.31 | 0.38 | 1.71 | 1.19 | 1.03 | 0.43 |
| TstdP | 37.21 | 8.06 | 49.68 | 31.69 | 29.12 | 10.69 |
| TtimeAf | 17.35 | 3.19 | 14.09 | 7.81 | 20.27 | 3.68 |
| TtimeMax_hNorm | 0.23 | 0.09 | 0.35 | 0.23 | 0.30 | 0.20 |
| TstdVAf | 6.41 | 2.05 | 5.62 | 3.37 | 8.52 | 2.14 |
| TstdVMax_hNorm | 0.27 | 0.09 | 0.43 | 0.27 | 0.36 | 0.17 |
| TransitionTypeToSit | 0.54 | 0.15 | 0.48 | 0.13 | 0.48 | 0.16 |
| lengthRangeVToSit | 20.39 | 12.34 | 20.27 | 17.52 | 20.62 | 16.02 |
| FrangeV | 1.09 | 0.26 | 1.08 | 0.32 | 0.78 | 0.26 |
| FtimeV | 88.96 | 13.79 | 82.21 | 9.81 | 92.82 | 15.46 |
| FjerkV | 0.01 | 0.00 | 0.01 | 0.00 | 0.01 | 0.00 |
| FstdV | 0.33 | 0.07 | 0.29 | 0.06 | 0.28 | 0.06 |
| FrangeAP | 1.10 | 0.19 | 1.01 | 0.17 | 0.95 | 0.20 |
| FtimeAP | 117.86 | 19.15 | 125.36 | 29.73 | 125.92 | 26.30 |
| FjerkAP | 0.01 | 0.00 | 0.01 | 0.00 | 0.01 | 0.00 |
| FstdAP | 0.37 | 0.06 | 0.34 | 0.06 | 0.31 | 0.06 |
| FrangeP | 163.75 | 31.97 | 218.81 | 137.49 | 135.78 | 51.74 |
| FtimeP | 107.11 | 22.24 | 109.74 | 23.85 | 114.67 | 24.11 |
| FjerkP | 1.58 | 0.46 | 2.04 | 1.31 | 1.22 | 0.59 |
| FstdP | 44.18 | 10.56 | 55.83 | 34.54 | 34.35 | 12.74 |
| FrangePitchAf | 5.47 | 0.87 | 5.91 | 0.73 | 5.28 | 1.02 |
| FrangePitchMax_hNorm | 0.46 | 0.13 | 0.59 | 0.20 | 0.45 | 0.15 |
| FstdPitchAf | 2.16 | 1.28 | 2.88 | 1.43 | 3.39 | 1.50 |
| FstdPitchMax_hNorm | 0.36 | 0.14 | 0.35 | 0.10 | 0.35 | 0.14 |
| TransitionTypeFromSit | 0.55 | 0.19 | 0.45 | 0.16 | 0.50 | 0.21 |
| lengthRangeVFromSit | 24.58 | 15.13 | 22.39 | 16.43 | 23.23 | 16.29 |
| gender | 0.63 | 0.49 | 0.67 | 0.48 | 0.26 | 0.44 |
| age | 78.66 | 4.35 | 77.89 | 4.99 | 64.77 | 9.48 |
| weightkg | 72.25 | 13.46 | 72.75 | 13.20 | 77.65 | 12.36 |
| height | 1.64 | 0.06 | 1.61 | 0.09 | 1.69 | 0.09 |

In the stand-to-sit transitions, significant differences were observed between the FL and the OA groups in the duration and STD calculated from the V axis (p=0.02, p=0.01, respectively) and in the range and STD calculated from the AP axis (p=0.03, p=0.04, respectively). The main differences between the FL and OA subjects were observed in features extracted from the histogram of the duration in which both the area under the curve of the cumulative distribution function (CDF) and presence of data in the highest peak were significant (p=0.008, p=0.002, respectively). The area under the curve of the CDF of the STD histogram in V was also significantly different between FL and OA subjects (p=0.02). In the sit-to-stand transition the presence of data in the highest peak in the histogram of the STD calculated from the Pitch axis was significantly different between FL and OA subjects.

Since a significant age difference was observed between the OA and PD groups (p<0.001), a logistic regression of the results had been performed to adjust for the age difference as can be seen in Table 3. The results in Table 3 indicate that many features relating to the transitions as calculated from signals received by the sensor were significantly different in the PD vs the OA subjects. The relationship between some of these features and key PD symptoms as assessed using UPDRS are shown in Table 4. As can be seen in Table 4, there is a significant correlation between some PD symptoms assessed using the UPDRS scale and values calculated for the PD patients from signals received by the sensor, thus indicating the ability to provide prognosis of such PD symptoms using such calculated values. The results in Table 4 are adjusted for weight, height, age and gender.

Machine learning results comparing the PD and OA groups is depicted in Table 5. As can be seen in Table 5, the machine learning results demonstrate that the PD and OA groups are distinguishable with high accuracy, specificity and sensitivity.

TABLE 3
Logistic regression of PD vs OA subjects
PD-OA adjusted to- type, weight, height, age, gender

| | | CI | | |
|---|---|---|---|---|
| | Unstandardized | Lower | Upper | |
| Feature | B | Bound | Bound | P |
| TrangeV | 0.058 | −0.084 | −0.031 | <0.0001 |
| TtimeV | 2.51 | −3.45 | 8.49 | 0.4 |
| TjerkV | −0.001 | −0.002 | 0 | 0.016 |
| TstdV | −0.006 | −0.017 | 0.005 | 0.274 |
| TrangeAP | 0.055 | 0.016 | 0.094 | 0.006 |
| TtimeAP | 11.52 | 4.35 | 18.68 | 0.002 |
| TjerkAP | 0.001 | 0 | 0.001 | <0.0001 |
| TstdAP | −0.017 | −0.03 | −0.005 | 0.008 |
| TrangeP | −16.73 | −25.21 | −8.25 | <0.0001 |
| TtimeP | 4.88 | 0.462 | 9.3 | 0.031 |
| TjerkP | −0.2 | −0.3 | −0.1 | <0.0001 |
| TstdP | −4.86 | −7.25 | −2.48 | <0.0001 |
| TtimeAf | 1.81 | 0.96 | 2.66 | <0.0001 |
| TtimeMax_hNorm | 0.056 | 0.014 | 0.098 | 0.01 |
| TstdVAf | 1.15 | 0.66 | 1.64 | <0.0001 |
| TstdVMax_hNorm | 0.077 | 0.04 | 0.113 | <0.0001 |
| TransitionTypeToSit | −0.074 | −0.108 | −0.041 | <0.0001 |
| lengthRangeVToSit | −3.83 | −7.26 | −0.39 | 0.29 |
| FrangeV | −0.17 | −0.23 | −0.1 | <0.0001 |
| FtimeV | 3.57 | −0.067 | 7.21 | 0.054 |
| FjerkV | −0.002 | −0.003 | −0.001 | <0.0001 |
| FstdV | −0.021 | −0.037 | −0.005 | 0.01 |
| FrangeAP | −0.065 | −0.11 | −0.018 | 0.007 |
| FtimeAP | 4.13 | −1.51 | 9.78 | 0.15 |
| FjerkAP | −0.001 | −0.001 | 0 | 0.11 |
| FstdAP | −0.023 | −0.038 | −0.008 | 0.003 |
| FrangeP | −20.31 | −31.84 | −8.78 | 0.001 |
| FtimeP | 7.46 | 2.05 | 12.87 | 0.007 |
| FjerkP | −0.27 | −0.4 | −0.14 | <0.0001 |
| FstdP | −5.78 | −8.71 | −2.85 | <0.0001 |
| FrangePitchAf | −0.22 | −0.45 | 0.01 | 0.06 |
| FrangePitchMax_hNorm | −0.007 | −0.042 | 0.029 | 0.71 |
| FstdPitchAf | 0.49 | 0.136 | 0.847 | 0.007 |
| FstdPitchMax_hNorm | 0.015 | −0.02 | 0.05 | 0.38 |
| TransitionTypeFromSit | −0.078 | −0.12 | −0.03 | 0.001 |
| lengthRangeVFromSit | −5.36 | −8.9 | −1.82 | 0.003 |

TABLE 4
Correlation between PD symptoms as assessed using UPDRS and values calculated
from continuous signals received from the body fixed sensor

| | | | CI | | |
|---|---|---|---|---|---|
| | | | Lower | Upper | |
| UPDRS prognosis | BFS Feature | beta | Bound | Bound | P |
| Falls in previous year | TrangeV | 0.11 | 0.005 | 0.016 | p < |
| | | | | | 0.0001 |
| UPDRS Part 3 item 14 off meds | TjerkV | −0.001 | −0.002 | 0 | 0.14 |
| UPDRS part 2 item 12 off | TstdV | 0.19 | 0 | 0.38 | 0.046 |
| medications | | | | | |
| Postural-instability and | | 0.011 | 0.002 | 0.019 | 0.017 |
| gait disturbance score | | | | | |
| UPDRS part 3 item 14 on | TjerkAP | 0.001 | 0 | 0.001 | 0.047 |
| medications | | | | | |
| UPDRS part 3 item 14 on | TjerkP | −0.152 | −0.29 | −0.007 | 0.041 |
| medications | | | | | |
| UPDRS part 2 item 11 off | TtimeMax_hNorm | −0.11 | −0.18 | −0.04 | 0.003 |
| medications | | | | | |
| Total UPDRS | TstdVAf | −0.06 | −0.11 | −0.005 | 0.033 |
| UPDRS part 2 item 11 Off meds | TstdMax_hNorm | −0.079 | −0.145 | −0.012 | 0.021 |
| Total UPDRS | FtimeV | 0.43 | −0.002 | 0.86 | 0.051 |
| UPDRS motor Score off | FstdV | 0.003 | 0 | 0.005 | 0.036 |
| Total UPDRS | TransitionTypeFromSit | −0.006 | −0.011 | 0 | 0.043 |

TABLE 5
Machine learning results comparing the PD and OA groups
(F# - number of features that were compared
using the machine learning algorithm)

| Iteration# = 20 | | | | | Naive- |
|---|---|---|---|---|---|
| Learn Size = 25 | | AdaBoostM1 | SVM | Bag | Bayes |
| Best result | Accuracy | 90.14 | 93.07 | 91.57 | 90.81 |
| | Specificity | 85.71 | 100 | 100 | 100 |
| | Sensitivity | 90.37 | 92.75 | 91.17 | 90.37 |
| | F# | 23 | 11 | 11 | 7 |
| Mean result | Accuracy | 88.07 | 90.59 | 88.39 | 89.36 |
| | Specificity | 72.42 | 76.54 | 78.26 | 74.98 |
| | Sensitivity | 88.99 | 91.39 | 88.98 | 90.21 |
| | F# | 17.15 | 15.3 | 12.75 | 9.5 |

### Example 2

### Identification of Freezing of Gait Using a Body-Fixed Sensor on a Subject's Body

Freezing of gait (FOG) is an episodic gait disturbance common in Parkinson's disease and serves as one measurement reflecting the severity of the disease. FOG occurs in approximately 60-80% of patients in the advanced stages of Parkinson's disease. FOG frequency and severity are extremely difficult to quantify and rely on patient or caregiver reports. In addition, identification of FOG in the early stages of the disease, when it is usually relatively rare and fleeting, may be even more difficult.

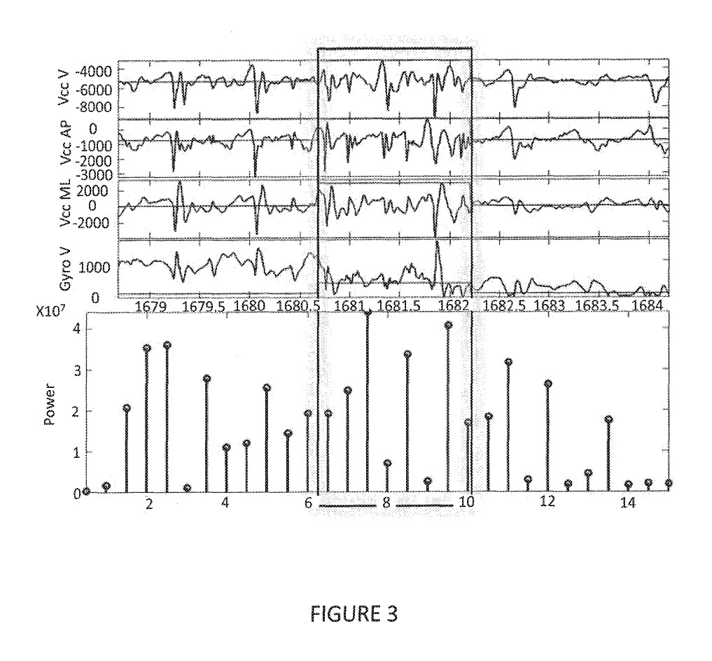

A single body-fixed sensor was placed on the lower back of a Parkinson's disease patient. As can be seen by the rectangle in FIG. 3, a FOG episode was identified by analyzing the different measured continuous signals including acceleration in the V, AP and ML axes.

### Example 3

### Identification of Impairment of Cognitive Functions in Parkinson's Disease Patients Using a Body-Fixed Sensor

Continuous signals corresponding to body movements of Parkinson's disease (PD) patients having high or low overall cognitive function (as measured by the Global Cognitive Score, GCS) were measured using a single body-fixed sensor worn on the lower back of the patients for 72 hours.

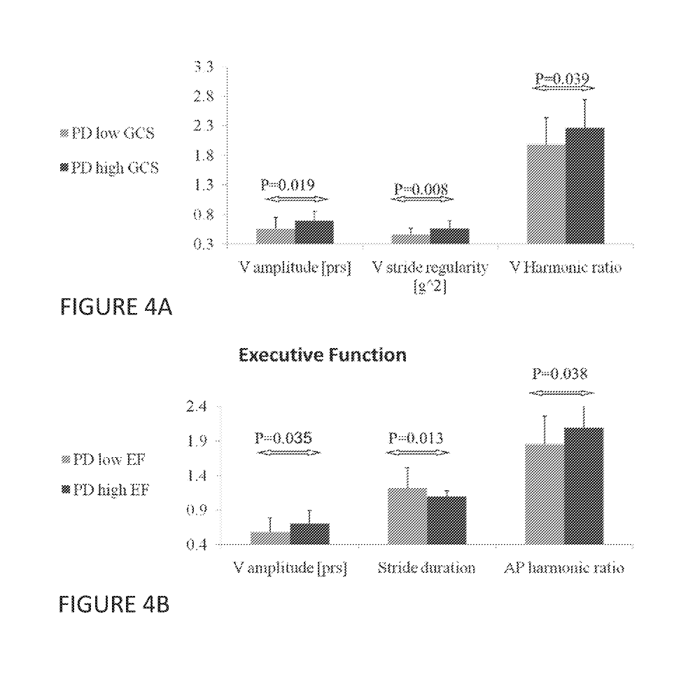

As can be seen in FIG. 4A, several values extrapolated from the signals are significantly different in PD patients having a low global cognitive score (PD low GCS) versus PD patients having a high global cognitive score (PD high GCS). Patients with high cognitive function demonstrated higher vertical amplitude (indicative of step variability), higher stride regularity and higher harmonic ratio (indicative of gait smoothness) with a significance of p=0.019, p=0.008 and p=0.039, respectively.

As can be seen in FIG. 4B, several values extrapolated from the signals are significantly different in PD patients having a low executive function (PD low EF) versus PD patients having a high executive function (PD high EF). Patients with high Executive Function demonstrated lower step variability (higher V amplitude and slope), lower step duration, as well as higher gait smoothness (higher AP Harmonic ratio) with a significance of p=0.035, p=0.013 and p=0.038, respectively.

These results demonstrate that measures reflecting different aspects of cognitive function can be extracted from continuous signals collected by body-fixed sensors.

### Example 4

### Measurement of Continuous Signals Using a Body-Fixed Sensor During the Night Enables the Analysis of Sleep Behavior

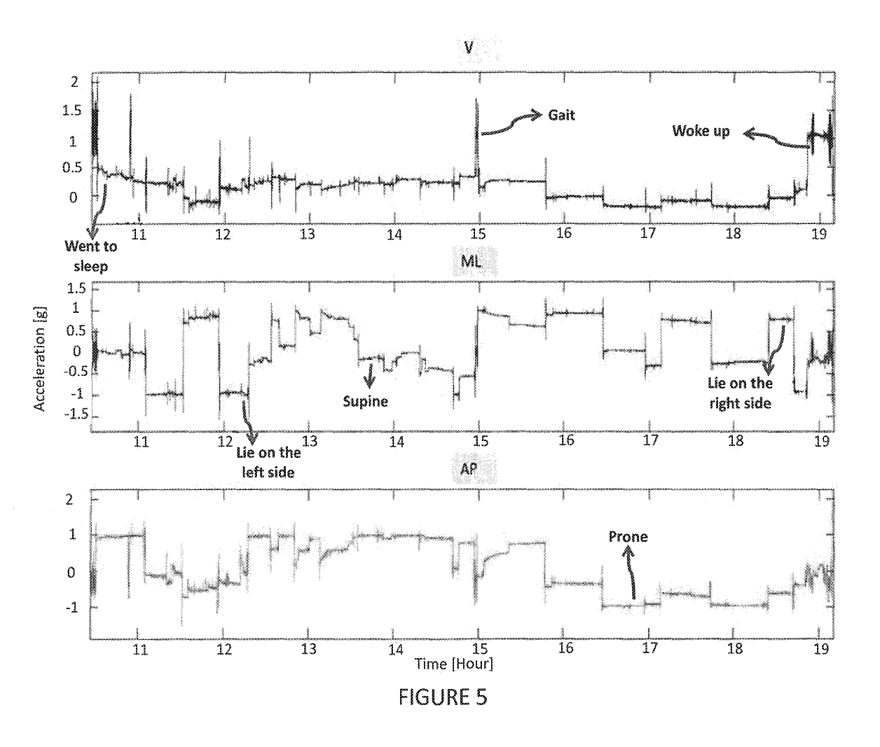

Continuous signals corresponding to the movements of a Parkinson's disease patient during sleep were measured by a body-fixed sensor fixed to the lower back of the patient. As can be seen in FIG. 5, the signals received from the body-fixed sensor were able to reveal several elements regarding the subject's sleep behavior, such as the amount of disruptions, the number of times the subject got up in the middle of the night and the amount and quality of movement during sleep. These results further demonstrate that a body-fixed sensor may be used for measurement of non-motor symptoms that are common in PD patients.

### Example 5

### Parkinson's Disease Patients can be Differentiated from Fallers Through Analysis of Continuous Measurements Received from a Body-Fixed Sensor During Sleep

Parkinson's disease patients (19 patients) and 13 non-PD elderly fallers wore a small device (AX3. Axivity; 6×21.5×31.5 mm, 9 g) that contained accelerometers on the lower back, approximately at the level of L4-5. Three channels were collected at 100 Hz each: vertical (V), medio-lateral (ML) and anterior posterior (AP) acceleration. The subjects wore the sensor for seven consecutive days. They were asked to wear it all day long while they performed their daily normal routine. A subject was classified as a faller if he/she reported at least two falls in the last 6 months.

The sleeping segments were extracted from the collected signals by checking the mean value of the acceleration in the V axis. Acceleration in the V axis which was around zero was interpreted to mean that the device is at 90 degrees and the subject was lying. The seven longest sleep segments were annualized under the assumption that these parts represent night sleep and not resting periods.

The night was split into two segments, asleep and awake. For the sleeping segments, the STD for each axis, the number of turns and the total sleep duration was found. For the wakening segments, the number of times that the subject was awake, the duration and the percent of activity from the total wakening time was measured. Furthermore, the gait segment was found during the 7 days.

By analyzing the results, a significant difference (p=0.006) was found between the fallers and the PD patients in the STD of the Medio-lateral (ML) axis during sleep.

### Example 6

### Continuous Signals Received Using a Body Fixed Sensor Enable Determining Disease Progression in Parkinson's Disease Patients

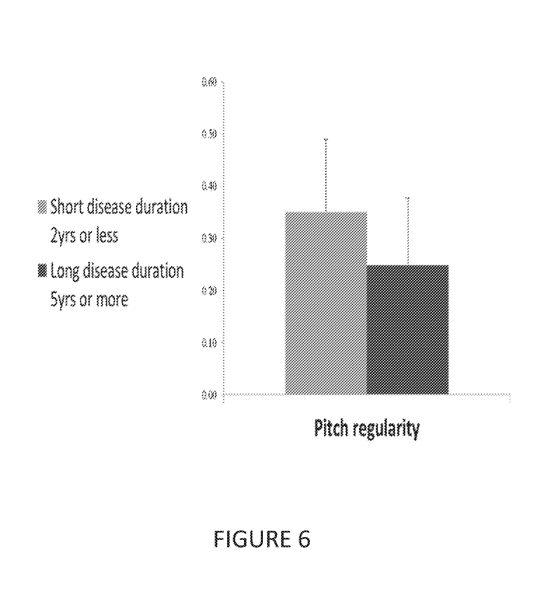

Body movements in two groups of Parkinson's disease patients were measured using a body-fixed sensor worn on the lower back. One of the groups included patients which were relatively recently diagnosed with the disease (Short disease duration, 2 yrs or less) and the other included patients who suffered from the disease for a longer period (Long disease duration, 5 yrs or more). As can be seen in FIG. 6, pitch regularity is lower in PD patients who suffered from the disease for a longer period. Pitch regularity is a measurement calculated from the acceleration signal of the body-fixed sensor. Pitch regularity may reflect the ability to generate forward movement that has been shown to deteriorate as PD progresses.

### Example 7

### Continuous Signals Received Using a Body Fixed Sensor Enable Determining Rigidity in Parkinson's Disease Patients

Arm swing was measured using a BFS (including an accelerometer and gyroscope) on bilateral wrists of patients with PD. Arm swing amplitude of the “most affected hand” was inversely correlated to the rigidity score of the patients on the UPDRS (r=−0.62, p=0.03).

### Example 8

### Continuous Signals Received Using a Body Fixed Sensor for Assessing Treatment Efficacy

In order to assess and evaluate treatment efficacy of PD patients, a wearable sensor was used to evaluate activity and motor response fluctuations in the patients before, during or after treatment.

In current health-care settings, self-reporting and repeated testing by a clinician are commonly used to assess motor response fluctuations; however, these approaches are intrusive and may not be sensitive to therapeutic changes.

### Methods:

A phase II randomized, placebo-controlled, double-blind trial with a Parkinson treatment was conducted on patients receiving PD treatment.

Twenty two patients with PD and motor response fluctuations (average age: 62.8±7.0 yrs, UPDRS motor score: 25.5±11.4) received their optimized, regular oral treatment (dose reductions permitted), and were randomized to adjunct therapy (treatment group/test group (TG), n=14) or placebo (Pacelbo group/control group (PG), n=8). The treatment group received an additional PD treatment (sub cutaneous (s.c.) administration of levodopa).

Subjects wore a 3D accelerometer on their lower back for 6 days before (pre) and for 6 days while receiving the treatment (during). In each 6 day period, the time spent walking and being inactive (i.e., lying or sitting) was determined. Wilcoxon signed ranked non-parametric tests evaluated the effects of the treatment (or placebo) on activity.

### Results:

Total time walked over the 6 days increased (p=0.048) from 652.9±266.6 min (pre) to 724.9±292.5 min (during) in the treatment group, but did not change in the Placebo group (p=0.249). Total inactive time decreased in the Treatment Group (pre: 8,380±3,044 min, during: 7,760±2,728 min; p=0.056), but not in the Placebo Group PG (p=0.401). The time spent walking between 4:00-5:00 AM, i.e., during sleep time, decreased (i.e., improved) in the TG (pre: 73.5±88.7 sec; during: 50.4±77.8 sec; p=0.011), but not in the PG (p=0.161). The time spent walking between 6-7 AM, i.e., at the start of the day, increased (i.e., improved) in the TG (pre: 104±112 sec; during: 162±125 sec; p=0.003), but not in the PG (p=0.263).

### CONCLUSIONS

The results presented above indicated that changes in the sensor-derived metrics suggest that the treated patients became more active during the day and more inactive at night, possibly a reflection of a reduction in motor response fluctuations.

Furthermore, the results obtained indicate that a continuously worn body-fixed sensor can provide objective assessment of activity that is sensitive to a pharmacologic intervention and can further be used to assess the treatment efficacy.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

1. A system for evaluating at least one of a motor function and a non-motor function affected by Parkinson's disease (PD) in a subject suffering from PD, the system comprising:
a body-fixed sensor configured to receive a signal corresponding to the subject's body movement; and
a processor configured to:
extract at least one movement segment in the body movement;
detect in said at least one extracted movement segment a plurality of measurements of at least one motor function affected by PD;
combine said plurality of measurements detected in said at least one extracted movement segment;
calculate, based on said signal, at least one value from said combination;
compare said at least one calculated value with at least one reference value; and
quantitatively evaluate the at least one of a motor function and a non-motor function in said subject based on said comparison.

2. The system of claim 1, wherein the non-motor function is selected from the group consisting of: a cognitive function, a sleep-behavior related function, a physiological symptom or combinations thereof.

3. (canceled)

4. The system of claim 1, wherein the evaluated function is selected from the group consisting of: fatigue, sleep-pattern, global cognitive score, executive function, attention, depressive symptoms, and a combination thereof.

5. (canceled)

6. The system of claim 1, wherein said plurality of values comprise vertical amplitude, stride regularity, harmonic ratio or any combination thereof.

7-14. (canceled)

15. The system of claim 1, wherein said processor is comprised in a mobile device.

16. The system of claim 1, wherein said one or more motor functions affected by Parkinson's disease are selected from the group consisting of: rigidity, movement amplitude, period of movement, movement speed, posture, postural control, bradykinesia, gait, balance, tremor, arm swing, trunk movement, sit-to-stand transition, stand-to-sit transition, sit-to-walk transition, walk-to-sit transition, turning, sitting, lying, sleep movements, fall and a combination thereof.

17. The system of claim 1, wherein said processor is configured to calculate values corresponding to at least two motor functions affected by Parkinson's disease.

18-19. (canceled)

20. The system of claim 1, wherein said at least one reference value is selected from the group consisting of: values obtained from a subject having Parkinson's disease, values obtained from a healthy subject, values obtained from said subject at an earlier time period, values corresponding to Parkinson's disease of a known severity level and a combination thereof.

21. The system of claim 1, wherein said processor is configured to calculate at least part of said at least one value based on a signal collected during a specific time-window.

22. (canceled)

23. The system of claim 1, wherein said sensor is configured to receive said signals consecutively for at least 1 hour.

24. A method for evaluating at least one of a motor function and a non-motor function affected by Parkinson's disease (PD) in a subject suffering from PD, the method comprising:
receiving a signal corresponding to the subject's body movement from a body-fixed sensor; and, via a processor:
extracting at least one movement segment in the body movement;
detecting in said at least one extracted movement segment a plurality of measurements of at least one motor function affected by PD;
combining said plurality of measurements detected in said at least one extracted movement segment;
calculating, based on said signal, at least one value from said combination;
comparing said at least one calculated value with at least one reference value; and
quantitatively evaluating the at least one of a motor function and a non-motor function of said subject based on said comparison.

25-38. (canceled)

39. A system for determining treatment efficacy for Parkinson's disease (PD) in a subject, the system comprising:
a body-fixed sensor configured to receive a signal corresponding to the subject's body movement, wherein the signal is received prior to, during, and/or after administration of said treatment; and
a processor configured to:
extract at least one movement segment in the body movement;
detect in said at least one extracted movement segment a plurality of measurements of at least one motor function affected by PD;
combine said plurality of measurements detected in said at least one extracted movement segment;
calculate, based on said signal, at least one value from said combination;
compare said at least one calculated value with at least one reference value; and
determine the efficacy of treatment based on said comparison.

40. The system of claim 39, wherein said at least one reference value is selected from reference values corresponding to said subject prior to administration of said treatment, reference values of the subject obtained at an earlier time point, reference values of a control group, reference values of subjects not afflicted with PD, reference values corresponding to Parkinson's disease of a known severity level or combinations thereof.

41. The system of claim 39, wherein the treatment comprises a therapeutic treatment (a drug), a physical exercise, cognitive training or any combinations thereof.

42-52. (canceled)

53. The system of claim 39, wherein said processor is further configured to determine a suitable treatment regime for the subject based on said comparison.

54-68. (canceled)

69. The system of claim 1, wherein said calculating includes at least one of a machine learning algorithm and logistic regression.

70. The system of claim 1, wherein said calculating includes performing a machine learning algorithm selected from an adaptive boost (AdaBoost) algorithm, a support vector machine (SVM) algorithm, a Bag algorithm, and a Naïve Bayes algorithm.

71. The system of claim 1, wherein said at least one movement segment is comprised of a portion of the body movement extracted during continuous measurements obtained while the subject is at least one of standing, sitting, walking, lying, transitioning from stand-to-sit, transitioning from sit-to-stand, transitioning from walk-to-sit, and transitioning from sit-to-walk.

72. The system of claim 1, wherein said plurality of measurements are indicative of at least one of step variability, gait smoothness, and step duration.

73. The system of claim 1, wherein said at least one of a motor function and a non-motor function is selected from a cognitive function and sleep behavior.

74. The system of claim 73, wherein said processor is configured to at least one of:
combine measurements of step variability and gait smoothness to evaluate cognitive function; and
combine measurements of step variability, step duration, and gait smoothness to evaluate executive function.

75. The system of claim 74, wherein said processor is configured to at least one of:
calculate vertical amplitude, stride regularity, and harmonic ratio to evaluate cognitive function; and
calculate vertical amplitude and slope, and harmonic ratio to evaluate executive function.
